(19) United States
(12) Patent Application Publication
(10) Pub. No.: US 2025/0211956 A1
(43) Pub. Date: Jun. 26, 2025

(54) SYSTEMS AND METHODS FOR PAIRING WIRELESS DEVICES WITHOUT A USER-ACTUATED MECHANISM

(71) Applicant: PLUME DESIGN, INC., Palo Alto, CA (US)

(72) Inventors: Zhicheng QIU, Cupertino, CA (US); William J. McFARLAND, Portola Valley, CA (US)

(21) Appl. No.: 19/073,553

(22) Filed: Mar. 7, 2025

Related U.S. Application Data
(63) Continuation of application No. 17/862,570, filed on Jul. 12, 2022, now abandoned.

(51) Int. Cl. H04W 4/80; A61B 5/00; G06F 3/01; G06F 3/0346; H04W 76/14; H04W 76/30

(52) U.S. Cl. CPC H04W 4/80; A61B 5/002; G06F 3/011; G06F 3/0346; H04W 76/14; H04W 76/30; G06F 2203/0384

(57) ABSTRACT

Wireless communication systems and methods are provided for receiving user inputs as request to change the state of a wireless communication link between first and second wireless devices. A device, according to one implementation, includes a casing and an internal sensor arranged within the casing. The internal sensor is configured to electrically receive user input that is unrelated to manipulation of user-actuated mechanical components moveable with respect to the casing. The device further includes a decoding device configured to decode the user input as a request to change the state of a wireless communication link with a secondary device.

### CROSS-REFERENCE TO RELATED APPLICATIONS

This application claims the benefit of priority from U.S. application Ser. No. 17/862,570, filed Jul. 12, 2022, which is incorporated herein by reference in its entirety.

### TECHNICAL FIELD

The present disclosure generally relates to systems and methods for pairing devices in a wireless network. More particularly, the present disclosure relates receiving user input for requesting the pairing process, whereby the user input does not involve a user-actuated mechanism, such as a button or switch.

### BACKGROUND

With the miniaturization of electronic devices over the years, various types of relatively small, wearable devices (e.g., watches, wrist bands, earbuds, headphones, etc.) have been improved to allow additional types of functionality. For example, in addition to earbuds and headphones having built-in electronics for changing volume and operation modes, wireless earbuds and headphones may also include Bluetooth technology to allow a user to listen to music from a mobile phone or other source without worrying about wires. Various types of control mechanisms (e.g., buttons, keys, switches, knobs, etc.) may be incorporated into the design of these wearable devices to allow the user to control other aspects as well.

Other types of portable or wearable devices may include emergency alert devices, which normally should be worn at all times to allow the wearer to have the ability to contact emergency personnel whenever necessary. There is a need in the field of medical alert systems to provide wearable emergency devices that can be worn at all times. Also, since these wearable items only benefit the wearer when they are actually being worn and since the wearer would normally want to keep these items with him or her at all times, there is also a need for manufacturers to produce these wearable medical items with certain design features and form factors that may be more comfortable for the wearer and which may be less obstructive to normal activities.

Many small wearable devices (e.g., rings, bracelets, wrist bands, necklaces, pendants, etc.) are simply decorative items and do not normally include user input mechanisms, such as buttons. However, these types of items are also being improved in recent years to provide additional functionality in wireless network environments.

Bluetooth-enabled devices, including those that can be worn by the user, usually are designed such that they constantly remain in a pairing mode to allow the device to pair with a mobile phone, access point, or other secondary device that may be used for connecting to the Internet. One problem with these constant-pairing scenarios, however, is that a nearby malicious device may be able to pair with the device and cause havoc. Therefore, there is also a need to control the Bluetooth (or Wi-Fi) pairing procedures, not only to prevent unwanted connection with malicious devices as mentioned here, but also to conserve battery life.

Also, the presence of buttons, switches, and other user-actuated mechanisms on wearable devices may normally lack a certain aesthetic quality, and there are currently very few options for hiding these user-actuated mechanisms. Therefore, there is another need to provide a more aesthetic solution for incorporating mechanisms to receive user input. Also, from a mechanical perspective, surface-mounted user-actuated mechanisms may suffer from the fact that they might not be completely waterproof or sealed against the environment, which can lead to problems with internal electrical circuitry. Also, conventional user-actuated mechanisms on small wearable devices may be difficult to move (e.g., depress, slide, toggle, etc.) and at times can be accidentally actuated. Also, it can be difficult at times to press or slide certain mechanisms adequately. There is therefore also a need to overcome these deficiencies of conventional systems.

### BRIEF SUMMARY

The present disclosure describes systems and methods for using a smart device that can be worn by a user. According to various implementations, a wearable device may include a casing and an internal sensor arranged within the casing. The internal sensor is configured to electrically receive user input that is unrelated to manipulation of user-actuated mechanical components moveable with respect to the casing. In addition, the wearable device further includes a decoding device configured to decode the user input as a request to change the state of a wireless communication link with a secondary device.

In some embodiments, the wearable device may further comprise a wireless communication device configured to wirelessly communicate with the secondary device. The wireless communication device may be configured to wirelessly communicate with the secondary device in accordance with Bluetooth communication protocols. The step of changing the state of the wireless communication link may include setting up the wireless communication link between the wearable device and the secondary device or tearing down the wireless communication link between the wearable device and the secondary device. In response to the decoding device decoding the user input as an “on” request, the wireless communication device is configured to open up the wireless communication link with the secondary device. In response to the decoding device decoding the user input as an “off” request, the wireless communication device is configured to close the wireless communication link with the secondary device.

Furthermore, the internal sensor may include one or more capacitance sensors configured to detect capacitance for determining whether or not a user is wearing the wearable device. As such, the decoding device may be configured to decode multiple changes in capacitance within a predetermined time frame as a user request to change the state of the wireless communication link with the secondary device.

The internal sensor may also include one or more wireless charging sensors configured to sense the presence of Near Field Communication (NFC) signals for determining whether or not the wearable device is placed on an NFC charger. As such, the decoding device may be configured to decode multiple changes in the presence of NFC signals within a predetermined time frame as a user request to change the state of the wireless communication link with the secondary device.

In some embodiments, the internal sensor may include one or more optoelectronic sensors. As such, the decoding device may be configured to decode movement of the wearable device with respect to a predetermined code image as a user request to change the status of the wireless communication link with the secondary device. The predetermined code image, for example, may be applied to a post of a Near Field Communication (NFC) charger. As such, the decoding device may be configured to decode movement of the wearable device in a counter-clockwise direction around the post as a user request to open up the wireless communication link. Also, the decoding device may be configured to decode movement of the wearable device in a clockwise direction around the post as a user request to close the wireless communication link. In some cases, the one or more optoelectronic sensors may include a Photoplethysmography (PPG) sensor for measuring blood pressure of the user.

Also, the internal sensor may include one or more accelerometers configured to detect movement or vibrations of the wearable device or an external tapping action on a surface of the casing. As such, the decoding device may be configured to decode patterns of movements, vibrations, and/or tapping actions as user requests to change the state of the wireless communication link.

The wearable device may be a ring, a watch, glasses, smart glasses, earbuds, headphones, a bracelet, a necklace, a pendant, or other similar device. The internal sensor may be configured such that it includes no button components, switch components, or user-actuated mechanical components that can be physically accessible by a user. In some embodiments, the wearable device may further comprise a microphone and/or a camera for receiving supplemental user input and/or may include a speaker, a tone generator, a light source, and/or an LED for providing confirmation feedback to the user. The secondary device in these embodiments may be an access point device, a mesh node, a repeater, a local controller, or the like.

### BRIEF DESCRIPTION OF THE DRAWINGS

The present disclosure is illustrated and described herein with reference to the various drawings. Like reference numbers are used to denote like components/steps, as appropriate. Unless otherwise noted, components depicted in the drawings are not necessarily drawn to scale.

FIG. 1 is a diagram illustrating a short range wireless communication sub-system, according to some embodiments of the present disclosure.

FIG. 2 is a diagram illustrating various Wi-Fi networks in which the wearable device of FIG. 1 is employed for wireless communication with various secondary devices, according to some embodiments.

FIG. 3 is a diagram illustrating a perspective view of a smart ring for wireless communication with a secondary device, according to some embodiments.

FIG. 4 is a diagram illustrating the smart ring of FIG. 3 being worn by a user, according to some embodiments.

FIG. 5 is a diagram illustrating a cross-sectional view of the smart ring of FIG. 3 showing antennas and internal electrical circuitry of the smart ring, according to some embodiments.

FIG. 6 is a schematic diagram illustrating the antennas and the electrical circuitry of the smart ring of FIG. 3 for communicating over one or more frequency bands, according to some embodiments.

FIGS. 7A and 7B are diagrams illustrating detectable actions of placing the smart ring on the user's finger and removing the smart ring from the user's finger, according to some embodiments.

FIGS. 8A and 8B are diagrams illustrating detectable actions of placing the smart ring on a post of a Near Field Communication (NFC) charger and removing the smart ring from the post of the NFC charger, according to some embodiments.

FIGS. 9A-9C are diagrams illustrating the use of an image code for enabling the entry of a user request to change the state of the wireless communication link with the secondary device, according to some embodiments.

FIG. 10 is a flow diagram illustrating a process for customizing a user request to change the state of a wireless communication link with a secondary device, according to some embodiments.

FIG. 11 is a flow diagram illustrating a process for changing the state of the wireless communication link between a wearable device and a secondary device, according to some embodiments.

### DETAILED DESCRIPTION

The present disclosure relates to networking systems and methods. In particular, the networks described in the present disclosure are related to wireless network in a local environment, such Wi-Fi network, Wireless Local Area Networks (WLAN), etc., and may also be applicable in Near Field Communication (NFC) networks. In some embodiments, wireless devices may communicate with each using Wi-Fi or Bluetooth communication protocols. More particularly, the embodiments of the present disclosure are directed to wearable devices (e.g., rings, watches, glasses, smart glasses, necklaces, pendants, earbuds, headphones, Virtual Reality (VR) goggles, heart monitoring devices worn on the wrist, etc.). Some of these wearable devices may include a small form factor, particularly a ring (or smart ring) that is worn on the finger of a user.

Specifically, by moving the wearable device in a particular sequence or pattern of motions (e.g., by moving a smart ring in a circular motion), user input can be obtained by utilizing sensors (e.g., accelerometers) built into the device. These motion may be interpreted as control input and/or may also be interpreted as a request to change the state of a wireless communication link between the wearable device and a secondary device (e.g., mobile phone, access point, gateway device, router, modem, etc.) in a wireless network.

There has thus been outlined, rather broadly, the features of the present disclosure in order that the detailed description may be better understood, and in order that the present contribution to the art may be better appreciated. There are additional features of the various embodiments that will be described herein. It is to be understood that the present disclosure is not limited to the details of construction and to the arrangements of the components set forth in the following description or illustrated in the drawings. Rather, the embodiments of the present disclosure may be capable of other implementations and configurations and may be practiced or carried out in various ways. Also, it is to be understood that the phraseology and terminology employed are for the purpose of description and should not be regarded as limiting.

As such, those skilled in the art will appreciate that the inventive conception, upon which this disclosure is based, may readily be utilized as a basis for the designing of other structures, methods, and systems for carrying out the several purposes described in the present disclosure. Those skilled in the art will understand that the embodiments may include various equivalent constructions insofar as they do not depart from the spirit and scope of the present disclosure. Additional aspects and advantages of the present disclosure will be apparent from the following detailed description of exemplary embodiments which are illustrated in the accompanying drawings.

### Wireless Communication Link

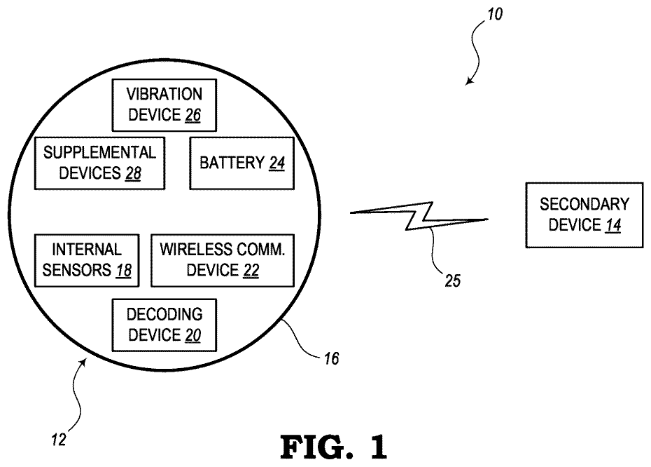

FIG. 1 is a diagram illustrating an embodiment of a short-range wireless communication sub-system 10. As illustrated, the short-range wireless communication sub-system 10 includes a wearable device 12 and a secondary device 14 configured to communicate wirelessly with one another. The secondary device 14 may be any suitable device that may be used in a wireless network, such as an access point device, modem, router, etc. The wearable device 12 can be any type of mobile device that can be worn by a user. For example, the wearable device 12 may have the form of a ring or other type of band around the finger of the user, a watch or other type of band around the wrist of the user, necklace, lanyard, or other type of strap or strip that hangs around the neck of the user, a pendant, glasses, etc. For simplicity, embodiments described in the present disclosure are directed to the wearable device 12 being a smart ring. However, it should be noted that the wearable device 12 may include other forms, as mentioned herein, and are not limited to just the ring form.

In some embodiments, the wearable device 12 may include a casing 16 or housing that is configured to surround and protect internal electrical circuitry. The internal circuitry may include one or more internal sensors 18, a processing device 20, a wireless communication device 22, and a battery 24. The one or more internal sensors 18 may include one or more accelerometers, one or more gyroscopic devices, one or more capacitance sensors, one or more NFC signal detection devices, one or more optoelectronic sensing devices, etc.

The processing device 20 may include decision functionality, such as a decoding module or decoding device for translating, decoding, or interpreting user input from the one or more internal sensors 18 from raw data into a user request or command. In this way, the way may purposefully move the wearable device 12 or provide a force to the wearable device in such a manner (e.g., using a sequence or pattern of motions, forces, taps, etc.) that the processing device 20 can decipher this user input as a request. In some cases, the request can be a request to perform control actions on the wearable device 12 or other external devices in the short-range wireless communication sub-system 10. However, according to the preferred embodiments of the present disclosure, the request may be interpreted as a request to change a state of a wireless communication link 25 between the wearable device 12 and the secondary device 14.

In some embodiments, the wearable device 12 may also include a vibration device 26 for providing haptic or tactile feedback to the user in response to receiving user input or for acknowledging the reception of a command or request. Also, in some embodiments, the wearable device 12 may further include one or more supplemental devices 28, such as one or more microphones, one or more cameras, one or more speakers or tone generating devices, and/or one or more light generating devices (e.g., LEDs).

It may be noted, therefore, that the wearable device 12 does not have any externally accessible buttons, keys, switches, slides, etc., which may be defined as conventional user-actuated mechanisms. Instead, the one or more internal sensors 18 are configured to detect presence or nearness (e.g., using capacitance sensing), detect NFC signals, detect motion (e.g., using accelerometers or gyroscopic devices), etc. In some cases, the one or more internal sensors 18 may also include optoelectronic devices for sensing image codes (e.g., barcodes, etc.). Thus, without moveable mechanisms (e.g., buttons, switches, etc.) on the surface of the casing 16, the wearable device 12 of the present disclosure can be more waterproof compared to conventional devices where a user manipulates surface-mounted user-actuated mechanisms. The wearable device 12 of the present disclosure may be referred to as a buttonless device, switchless device, etc.

According to one embodiment, the internal sensor 18 may include a capacitance sensor to detect if the wearable device 12 (e.g., ring) is on the finger of the user or is on an NFC charger or on some other component with a post. Also, the internal sensor 18 may include a wireless charger sensor (e.g., NFC sensor) for determining if the wearable device 12 is on a post of an NFC charger. Furthermore, the internal sensor 18 may include an optoelectronic sensor (e.g., Photoplethysmography (PPG) sensor) for detecting LED reflection. Various conditions (e.g., on finger, off finger, on post, off post, etc.) may be decoded as requests (e.g., factory set or customized) to pair the wearable device 12 with the secondary device 14 (i.e., set up the wireless communication link 25 between the two) or to break down or close the wireless communication link 25.

Regarding the application of one or more capacitance sensors, the internal sensors 18 may detect if the ring is on the user's finger. For example, the status or condition of the ring on the finger can be indicated with a binary 1, while the status or condition of the ring off the finger can be indicated with a binary 0. The processing device 20 may be configured to use any suitable on/off sequence (e.g., 010101, or off-on-off-on-off-on), within a limited time, to recognize the intention to enter user input for requesting that the wireless communication link 25 is turned on to pair the wearable device 12 with the secondary device 14. In other words, the user may repeatedly move the wearable device 12 (e.g., ring) on and off the user's finger within a short amount of time. The processing device 20 or decoding device may interpret this as a request to set up the wireless communication link 25 (e.g., turning on a Bluetooth pairing mode).

Similarly, an on/off sequence of, say, “101010” may be interpreted as a user request to turn off the Bluetooth pairing mode or close or break down the wireless communication link 25. For example, repeated on/off patterns may be analyzed by the processing device 20, where ending in a one means that the user is requesting to turn on the wireless communication link 25 and ending in a zero means that the user is requesting to turn off the wireless communication link 25.

The wearable device 12 may also have a similar way of talking with an NFC charger or case. Again, if the wearable device 12 (e.g., ring) is on the NFC charger, this may be indicated by a binary 1 and, if the wearable device 12 is off the NFC charger, this may be indicated by a binary 0. The processing device 20 or decoding device may be configured to operate in a way that is similar to the “finger” example above. In other embodiments, the opposite state of the wireless communication link 25 may be maintained with respect to the on or off condition. For example, when the wearable device 12 is on a post of the NFC charger (e.g., binary one), the wireless communication link 25 may be turned off, whereby, when the wearable device 12 is off the post of the NFC charger (e.g., binary zero), the wireless communication link 25 may be turned on.

The specific codes, sequences, or patterns of conditions may, in effect, be equivalent to a user's action of manipulating a conventional button for turning on or turning off a Bluetooth or Wi-Fi pairing. The codes, sequences, or patterns may be customized user-defined codes or factory-set codes.

Furthermore, a more complex way of entering user input is to make use of one or more optoelectronic sensors, such as the PPG sensors. In this case, the optoelectronic sensors may be configured to read an image code (e.g., barcode or other type of visually detectable code). In the example of a ring, the image code may be printed or applied in any other suitable manner to a post. For example, the post may be a charging pole (or rod) of an NFC charger or, in other embodiments, may simply be a post used exclusively for the purpose described herein. In the example of other types of wearable devices, the image code can be applied to any suitable surface.

Then, when the wearable device 12 (e.g., ring) is placed on the post (or move close to the image code), the wearable device 12 may be configured to turn on a light associated with the sensor for a short time. During this time, the user can twist the wearable device 12 (e.g., ring) around the post (or move the wearable device 12 is another suitable manner with respect to the image code). A photodetector of the wearable device 12 may be configured to read the image code (e.g., similar to scanning a barcode). Arbitrary image codes may be used for these predefined purposes. In some embodiments, a counter-clockwise twisting of the ring may represent a user request to open up the wireless communication link 25 (i.e., turn on the Bluetooth pairing), while a clockwise twisting of the ring may represent a user request to close the wireless communication link 25 (i.e., turn off the Bluetooth pairing).

Again, the one or more internal sensors 18 may include one or more accelerometers for measuring force, acceleration, vibration, movement, motion, etc. A particular tap pattern on the casing 16 of the wearable device 12 may be interpreted as a request to pair with the secondary device 14. In response to decoding this user input, the processing device 20 can be configured to cause the wireless communication device 22 to open up the Bluetooth pairing or wireless communication link 25 with the secondary device 14 to go into the pairing mode. The tap pattern could be user-defined. In this case, the user-defined pattern may prevent others (e.g., malicious strangers) from knowing a pattern and using the user's wearable device 12 without permission. In some case, a factory-based pattern may be used, such as Morse code using various combinations of quick taps and long taps.

In addition to tap patterns, the accelerometers may be used to measure motions. For example, moving the wearable device 12 in a particular pattern could be interpreted as a user request to pair, which can be following by the action of causing the wireless communication device 22 to go into the pairing mode. In some examples, the movement pattern may include moving the wearable device 12 in a figure-eight shape, making repeated circular motions in one or multiple directions, etc.

Additionally, customized patterns may be performed to mimic certain actions that a user may take at certain times when it may be desirable to turn on the wireless communication link. For example, one pattern could mimic the user's action when he or she might typically want to turn on the Bluetooth pairing, such as when the user comes home. The action pattern may include movements of the fingers, hands, etc. that the user might make when he or she first gets home, such as the action of turning a key in a lock to unlock a front door to the home and pushing the door open.

In accordance with some embodiments, the wearable device 12 may further include the supplemental devices 28. Some devices may have a microphone. The microphone could be used to interpret a speech phrase, such as “ring, go into pairing mode.” The microphone could also listen for a user tapping on a table top or other surface in a particular pattern (e.g., Morse code). Another input device of the supplemental devices 28 may include a camera. The camera could detect a particular hand gesture (e.g., waving a hand or finger, repeating gestures, thumb up and thumb down patterns, etc.). The camera may also be configured to scan an image or code (e.g., barcode, QR code, etc.) related to user input, which can result in the wearable device 12 going into pairing mode when these are detected.

The supplemental devices 28 may also include output devices. For example, the wearable device 12 may include a speaker or tone generator. The speaker or tone generator might provide an audio signal to confirm that the user request to enter pairing mode has been received or recognized or that the Bluetooth pairing has been opened in response to the user request. Another output device may be an LED or other light source. The light source may shine a particular pattern or color to indicate that the user request has been received or that the device has gone into pairing. Different blinking patterns and colors may be used to indicate different things.

Again, the wearable device 12 may include any type of device, such as a ring, a watch, glasses, smart glasses, VR goggles, handsfree headsets, headphones, earbuds, bracelets, necklace, pendant worn on a necklace, device worn on the wrist or chest (e.g., heart monitor), etc. In some embodiments, the wearable device 12 may also be associated with a case for storing the device when not in use and/or for recharging batteries on the wearable device 12. This may be a buttonless devices, as mentioned herein, where there are no buttons for changing the state of wireless communication link 25. In some cases, the wearable device 12 may include other types of buttons for purposes other than for changing the pairing. In some embodiments, the secondary device 14 might be a computer, tablet, laptop, mobile phone, modem, router, access point, stereo system, car stereo system, wireless speakers, smart lock, etc.

### Wi-Fi Networks

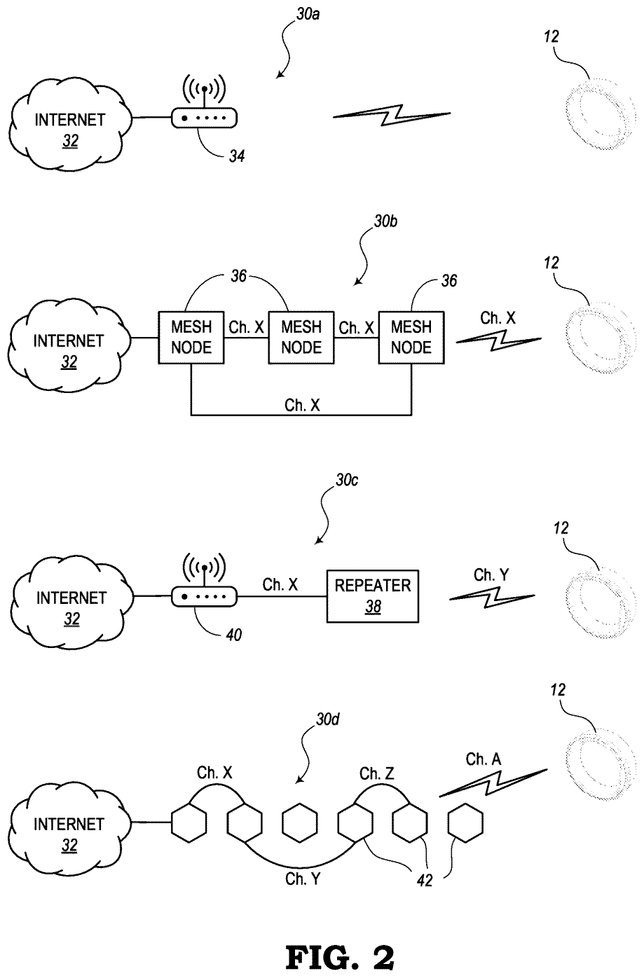

FIG. 2 is a diagram illustrating various Wi-Fi networks 30a, 30b, 30c, 30d in which the wearable device 12 shown in FIG. 1 can be employed for wireless communication with various secondary devices, which may be same as or similar to the secondary device 14 described with respect to FIG. 1. In this regard, the secondary devices are labelled 34, 36, 38, and 42 in FIG. 2.

The Wi-Fi networks 30a, 30b, 30c, 30d may be local networks in a home or office for connectivity or access to the Internet 32. The Wi-Fi networks 30 can operate in accordance with the IEEE 802.11 protocols and variations thereof. The Wi-Fi networks 30 are deployed to provide coverage in a physical location (e.g., home, business, store, library, school, park, etc.). The different topologies of the Wi-Fi networks 30 may provide different scopes of physical coverage. As described herein, the Wi-Fi network 30 can be referred to as a network, a system, a Wi-Fi network, a Wi-Fi system, a cloud-based Wi-Fi system, etc. The Wi-Fi networks 30 may utilize one or more access points 34. In some embodiments, the Wi-Fi networks 30 may also or alternatively include mesh nodes 36, repeater 38, Wi-Fi pods or modules, and/or other components, which can be referred to as nodes, access points, Wi-Fi nodes, Wi-Fi access points, etc.

One objective of nodes is to provide network connectivity to the wearable device 12, which in some cases can be referred to as client devices, user equipment, user devices, clients, Wi-Fi clients, Wi-Fi devices, etc. It may also be noted that other types of Wi-Fi client devices, which may be configured for connectivity with the Internet 32, can be mobile devices, tablets, computers, consumer electronics, home entertainment devices, televisions, Internet of Things (IoT) devices, or any network-enabled devices.

The Wi-Fi network 30a includes a single access point 34, which can be a single, high-powered access point and may be centrally located to serve all smart rings 10 and Wi-Fi client devices in a location. Of course, a typical location can have several walls, floors, etc. between the single access point 14 and the Wi-Fi client devices (e.g., wearable device 12). Plus, the single access point 14 operates on a single channel (or possible multiple channels with multiple radios), leading to potential interference from neighboring systems.

The Wi-Fi network 30b is a Wi-Fi mesh network that solves some of the issues with the single access point 34 by having multiple mesh nodes 36, which distribute the Wi-Fi coverage. Specifically, the Wi-Fi network 30b operates based on the mesh nodes 36 being fully interconnected with one another, sharing a channel (e.g., channel X) between each of the mesh nodes 36 and the wearable device 12 and/or other Wi-Fi client devices. That is, the Wi-Fi network 30b may be a fully interconnected grid, sharing the same channel, and allowing multiple different paths between the mesh nodes 36 and the wearable device 12 and/or Wi-Fi client devices. However, since the Wi-Fi network 30b uses the same backhaul channel, every hop between source points divides the network capacity by the number of hops taken to deliver the data. For example, if it takes three hops to stream a video to a Wi-Fi client device, the Wi-Fi network 30b may be left with only one-third the capacity.

The Wi-Fi network 30c includes the access point 34 coupled wirelessly to one or more Wi-Fi repeaters 38. The Wi-Fi network 30c with the Wi-Fi repeaters 38 may be configured with a star topology, whereby there is at most one Wi-Fi repeater 38 between the access point 34 and the Wi-Fi client device (or wearable device 12). From a channel perspective, the access point 34 can communicate to the Wi-Fi repeater 38 on a first channel (e.g., Channel X) and the Wi-Fi repeater 38 can communicate to the wearable device 12 and/or Wi-Fi client devices on a second channel (e.g., Channel Y). The Wi-Fi network 30c solves the problem with the Wi-Fi mesh network of requiring the same channel for all connections by using a different channel or band for the various hops to prevent slowing down the Wi-Fi speed. It may be noted that some hops may use the same channel or band, but this is not required. One disadvantage of the repeater 38 is that it may have a different Service Set Identifier (SSID) from the access point 34, which is effectively different Wi-Fi networks from the perspective of the Wi-Fi client devices.

Despite Wi-Fi's popularity and ubiquity, many consumers still experience difficulties with Wi-Fi. The challenge of supplying real-time media applications is that it puts increasing demands on the throughput, latency, jitter, and robustness of Wi-Fi. Studies have shown that broadband access to the Internet through service providers is up 99.9% of the time at high data rates. However, despite the Internet arriving reliably and quickly to the edge of consumer's homes, simply distributing the connection across the home via Wi-Fi is much less reliable, leading to poor user experience.

Several issues that prevent conventional Wi-Fi systems from performing well include i) interference, ii) congestion, and iii) coverage. Regarding interference, the growth of Wi-Fi has come with the growth of interference between different overlapping Wi-Fi networks. When two networks within range of each other carry high levels of traffic, they tend to interfere with each other, reducing the throughput that either network can achieve. Regarding congestion, within a single Wi-Fi network, there may be several communications sessions running. When several demanding applications are running, such as high-definition video streams, the network can become saturated, leaving insufficient capacity to support the video streams. Regarding coverage, Wi-Fi signals attenuate with distance and when they travel through walls and other objects. In many environments, such as large residences, reliable Wi-Fi service cannot be obtained in all rooms. Even if a basic connection can be obtained in all rooms, many locations will have poor performance due to a weak Wi-Fi signal. Various objects in a residence (e.g., walls, doors, mirrors, people, and general clutter) all interfere and attenuate Wi-Fi signals leading to slower data rates.

Two general approaches have been tried to improve the performance of conventional Wi-Fi systems, as illustrated in the Wi-Fi networks 30b, 30c. The first approach (i.e., Wi-Fi network 30a) is to simply build more powerful single access points 34, in an attempt to cover a location with stronger signal strengths, thereby providing more complete coverage and higher data rates at a given location. However, this approach is limited by both regulatory limits on the allowed transmit power, and by the fundamental laws of nature. The difficulty of making such a powerful access point 34, whether by increasing the power, or increasing the number of transmit and receive antennas, grows exponentially with the achieved improvement. Practical improvements using these techniques lie in the range of 6 to 12 dB. However, a single additional wall can attenuate by 12 dB. Therefore, despite the huge difficulty and expense to gain 12 dB of the link budget, the resulting system may not be able to transmit through even one additional wall. Any coverage holes that may have existed will still be present, devices that suffer poor throughput will still achieve relatively poor throughput, and the overall system capacity will be only modestly improved. In addition, this approach does nothing to improve the situation with interference and congestion. In fact, by increasing the transmit power, the amount of interference between networks actually goes up.

Other approaches (i.e., Wi-Fi network 30b, 30c) is to use a mesh of Wi-Fi devices 36 or repeaters 38 to repeat the Wi-Fi data throughout a location, as illustrated in the Wi-Fi networks 30b, 30c. These approaches are fundamentally better approaches to achieving better coverage. By placing even a single repeater 38 in the center of a house, the distance that a single Wi-Fi transmission must traverse can be cut in half, halving also the number of walls that each hop of the Wi-Fi signal must traverse. This can make a change in the link budget of 40 dB or more, a huge change compared to 6 dB to 12 dB type improvements that can be obtained by enhancing a single access point 34 as described above. Mesh networks have similar properties as systems using Wi-Fi repeaters 38. A fully interconnected mesh adds the ability for all the mesh nodes 36 to be able to communicate with each other, opening the possibility of packets being delivered via multiple hops following an arbitrary pathway through the network.

The Wi-Fi network 30d includes various Wi-Fi devices 42 that can be interconnected to one another wirelessly (e.g., Wi-Fi wireless backhaul links) or wired, in a tree topology, where there is one path between the Wi-Fi client device (or wearable device 12) and a gateway (e.g., a Wi-Fi device 42 connected to the Internet 32), but which allows for multiple wireless hops unlike the Wi-Fi repeater network and multiple channels unlike the Wi-Fi mesh network. For example, the Wi-Fi network 30d can use different channels/bands between Wi-Fi devices 42 and between the wearable device 12 and/or Wi-Fi client device (e.g., Ch. X, Y, Z, A), and, also, the Wi-Fi system 30d does not necessarily use every Wi-Fi device 42, based on configuration and optimization. The Wi-Fi network 30d is not constrained to a star topology as in the Wi-Fi repeater network 30c which at most allows two wireless hops between the Wi-Fi client device (or wearable device 12) and a gateway or access point 34. Wi-Fi is a shared, simplex protocol meaning only one conversation between two devices can occur in the network at any given time, and if one device is talking, the others need to be listening. By using different Wi-Fi channels, multiple simultaneous conversations can happen simultaneously in the Wi-Fi network 30d. By selecting different Wi-Fi channels between the Wi-Fi devices 22, interference and congestion can be avoided or minimized.

Of note, the systems and methods described herein contemplate operation through any of the Wi-Fi networks 30, including other topologies not explicated described herein. Also, if there are certain aspects of the systems and methods which require multiple nodes in the Wi-Fi network 30, this would exclude the Wi-Fi network 30a.

### Smart Ring

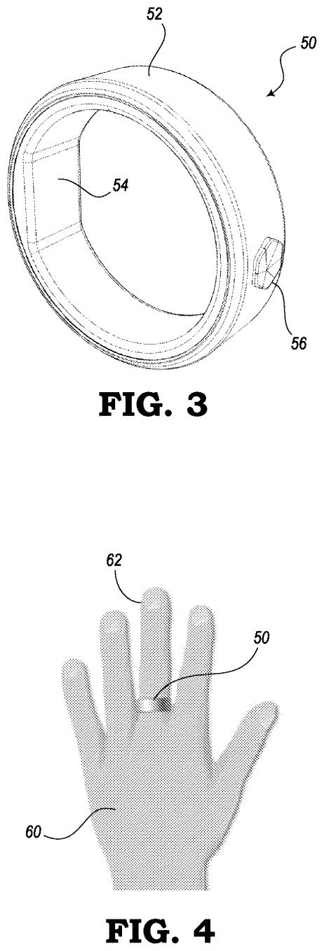

FIG. 3 is a diagram illustrating a perspective view of a smart ring 50 (e.g., wearable device 12) for wireless communication with a secondary device (e.g., secondary device 14). As shown in FIG. 3, the smart ring 50 includes a band 52 that is configured to fit around a user's finger (or thumb). In this embodiment, the smart ring 50 may also include a width expanded portion 54, which fills in a portion of an inside curve of the band 52. The width expanded portion 54 may have a substantially planar surface facing the middle of the band 52. The width expanded portion 54 may be helpful for keeping the smart ring 50 in a set orientation around the user's finger, the significance of which may be understood from the description below. Also, the smart ring 50 may include a raised feature 56. In some embodiments, the raised feature 56 may simply be a decorative element or a logo. In other embodiments, however, the raised feature 56 may include functional elements, such as a camera, microphone, speaker, etc.

In some embodiments, the smart ring 50 may include a titanium finish, may be light weight and have a slim profile (e.g., less than 3.5 mm thick and 8 mm wide). Also, the smart ring 50 may be water resistant, have a one-week rechargeable battery, and may include any various size. In some embodiments, the smart ring 50 can include various sensors, such as a 14-bit Photoplethysmography (PPG) sensor, a three-axis accelerometer, etc. The smart ring 50 can be configured to measure vitals, such as heart rate, heart rate variability, sleep patterns, activity levels, falls, and the like.

The smart ring 50 may include one or more accelerometers (e.g., three-axis accelerometer) or other suitable devices for detecting movement and forces related to motion of the user's hand or finger on which the smart ring 50 is worn. Using these movement detecting devices, the smart ring 50 can interpret specific sequences or patterns of taps or other motions. For example, based on predetermined or customized movement patterns, the smart ring 50 may interpret these “hand signals” from the user as control commands or instructions. For example, the commands may include instructions for turning on or turning off certain electrical devices (e.g., lights, lamps, fans, televisions, kitchen appliances, ovens, stoves, etc.), controlling the volume or level of speakers, fans, analog devices, or other electrical devices having multiple or variable settings, dialing a phone number of a mobile phone, contacting emergency personnel, or other action.

Thus, by performing certain movement profiles, the smart ring 50 is configured to communicate control commands to a controller that is equipped to control one or more electronic devices in a local network. According to other embodiments, instead of a ring, the device worn by a user and used for entering control commands may be another type of wearable item, such as a bracelet, arm band, ankle band, etc.

FIG. 4 is a diagram illustrating the smart ring 50 of FIG. 3 being worn on a user's hand 60, such as on a finger 62. It may be noted that the smart ring 50 can be worn on any finger, thumb, or even a toe of the user. In some cases, it may be beneficial to the user to wear the smart ring 50 on a finger that has good mobility (e.g., pointer finger, middle finger, etc.), thereby allowing the user to move the smart ring 50 in a controlled manner to more easily communicate motion information that can be properly interpreted.

The smart ring 50 may be configured to wirelessly communicate at short range to various devices, such as a control device incorporated in a local network (e.g., Wi-Fi network). The control device, in some embodiments, may be a stand-alone device, an access point device of a Wi-Fi system, a modem, a switch, a network node, a gateway device, a Bluetooth beacon device, a hub device, a mobile phone, etc. For example, when positioned near such a control device, the smart ring 50 and control device may be configured to operate within a first frequency band (e.g., Bluetooth frequencies) to enable communication therebetween. In response to receiving control commands and/or movement information from the smart ring 50, the control device may be configured to control one or more electrical devices in the local network or within a certain setting or periphery.

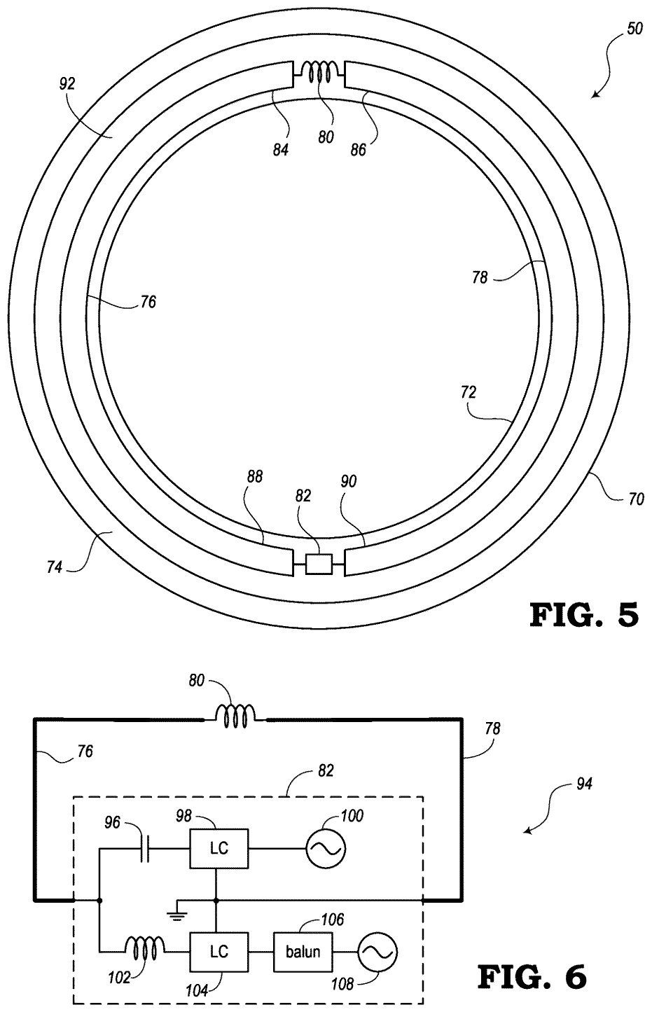

FIG. 5 is a diagram illustrating a cross-sectional view of an embodiment of the smart ring 50 of FIG. 3. The smart ring 50 includes antennas and internal electrical circuitry for enabling communication with other components in a network. Also, as suggested above, the electrical circuitry of the smart ring 50 may include one or more accelerometers or other suitable movement detection devices for detecting the motion of the user's finger 62 or smart ring 50.

As illustrated in FIG. 5, the smart ring 50 includes an outer surface 70 that may usually be visible when it is worn on a user's finger 62 (not shown in FIG. 4) and an inner surface 72 that may usually be in contact with the user's finger 62. An outer portion of the smart ring 50 may include a metallic layer 74, which may include the outer surface 70 in some embodiments.

Also, the smart ring 50 includes a first antenna component 76 and a second antenna component 78. The first and second antenna components 76, 78, in combination, may form a ring or tube having a relatively narrow width (e.g., measured from an outer surface to an inner surface as shown in FIG. 4) and a relatively narrow depth (e.g., measured into the page). In some embodiments, the depth of each of the first and second antenna components 76, 78 may have a dimension that is greater than its width.

Furthermore, the smart ring 50 includes a first electrical circuit 80 and a second electrical circuit 82. The first electrical circuit 80 is configured to electrically connect a first end portion 84 of the first antenna component 76 with a first end portion 86 of the second antenna component 78. Also, the second electrical circuit 82 is configured to electrically connect a second end portion 88 of the first antenna component 76 with a second end portion 90 of the second antenna component 78.

Conventional smart rings normally do not allow operation within two separate frequency bands. However, according to the various embodiments of the present disclosure, various antenna components of the smart ring 50 include specific physical characteristics and electrical circuitry that enable operation at two different frequency band. This allows the smart ring 50 to pair with a mobile device to enable operation within the first frequency band (e.g., Bluetooth) while also allowing the smart ring 50 to pair with a Point-of-Sale (POS) machine to enable operation within the second frequency band (e.g., NFC). In particular, antenna portions, as described below, may be configured to be fully embedded in a normal-sized ring. These antenna portions may include, for example, the electrically conductive battery casing and also a conductive trace or film on a Flexible Printed Circuit (FPC) or other suitable flexible board that can be embedded within the normal-sized ring. By using these components, which may already be needed for wireless communication, it may be possible to minimize the extra number of parts and circuitry to conserve space within the outer shell of the smart ring 50.

FIG. 6 is a schematic diagram illustrating an embodiment of an antenna circuit 94 of the smart ring 50 for communicating over one or more frequency bands. The antenna circuit 94 includes the first electrical circuit 80, the second electrical circuit 82, and the first and second antenna components 76, 78 connected between the first and second electrical circuits 80, 82. According to some embodiments, the first electrical circuit 80 may simply include an inductor configured to act like an open circuit at higher frequencies (e.g., Bluetooth and Wi-Fi frequencies) and act like a short circuit at lower frequencies (e.g., NFC frequencies).

As shown in the embodiment of FIG. 6, the second electrical circuit 82 includes a first set of components 96, 98, 100 configured for operation at the higher frequency range (e.g., Bluetooth, Wi-Fi) and a second set of components 102, 104, 106, 108 configured for operation at the lower frequency range (e.g., NFC). The first set of components includes a frequency blocking device 96 (e.g., series-connected capacitor), a higher-frequency matching circuit 98 (e.g., a combination of series-connected and shunt-connected inductors and capacitors), and a higher-frequency radio transceiver 100. The second set of components includes a higher-frequency choke or choke inductor 102 (e.g., a series-connected inductor or ferrite bead), a lower-frequency matching circuit (e.g., combination of series-connected and shunt-connected capacitors), a lower-frequency balun 106, and a lower-frequency radio transceiver 108. The matching circuits 98, 104 may be connected to ground and the radio transceivers 100, 108 may also be connected to ground.

To design an efficient antenna according to antenna theory, the length of the antenna is typically one fourth, one half, or one whole wavelength of the frequency of operation. For example, at a Bluetooth or Wi-Fi frequency of about 2.4 GHz, the wavelength is about 120 mm. At an NFC frequency of about 13.56 MHz, the wavelength is about 22 m (i.e., 22,000 mm). Other similar wavelengths may be applicable at other Bluetooth or Wi-Fi frequencies (e.g., about 2.4000 GHz to about 2.4835 GHZ) or at other NFC frequencies (e.g., about 12.66 MHz to about 14.46 MHz).

Rings typically vary in diameter from about 12 mm to about 22 mm and typically vary in internal circumference from about 49 mm to about 72 mm. Even the largest ring sizes are well below the typically minimum required diameter dimension of one-fourth of the wavelength (i.e., 120 mm/4=30 mm at Bluetooth frequency). Even if the entire ring is used for antenna volume it still would not be enough. This does not even include all the other parts, like battery, photo diode sensors, RF board, chips, etc.

Typical designs on the market use chip antennas that are a few mm by a few mm in size, but which require dedicated antenna volume that is already scarce. In addition, chip antennas have low performance as they typically rely on PCB ground currents that are weak in ring size (e.g., due to the small size of the PCB itself). Nevertheless, the configuration of the first and second antenna components 26, 28 as described with respect to the embodiments of the present disclosure allows the circumference dimension to be utilized in a specific way to enable operation in both frequency bands. Operation is contemplated in both frequency bands simultaneously. For example, the NFC band could be used for charging while the Bluetooth band is used for accessing another Bluetooth device, e.g., a phone, or Wi-Fi access point. Another example can include using the ring for payment (NFC) while maintaining a connection to a phone (Bluetooth).

In response to the first and second electrical circuits 80, 82 being configured in a first state, the first antenna component 76 and second antenna component 78 are configured to operate within a first frequency band (e.g., Bluetooth, Wi-Fi). In response to the first and second electrical circuits 80, 82 being configured in a second state, the first antenna component 76 and second antenna component 78 are configured to operate within a second frequency band (e.g., NFC). Also, in response to the first and second electrical circuits 80, 82 being configured in the first state, the first antenna component 76 and second antenna component 78 are configured in a dipole antenna arrangement (e.g., when the inductor 80 acts as an open circuit). In response to the first and second electrical circuits 80, 82 being configured in the second state, the first antenna component 76 and second antenna component 78 are configured in a loop antenna arrangement (e.g., when the inductor 80 acts as a short circuit).

According to some embodiments, operation within the first frequency band may enable pairing with a smart phone (or mobile device) and operation within the second frequency band enable pairing with a Point of Sale (POS) device. The antenna system may further include a battery configured to power one or more of the first and second electrical circuits 76, 78. The battery may include an outer metal casing that forms at least a portion of the first antenna component 76. The antenna system may also include a Near-Field Communication (NFC) charger. The NFC charger may be configured to create a magnetic field for charging the battery. The first frequency band may include one or more channels in a Bluetooth or Wi-Fi frequency band ranging from about 2.4000 GHz to about 2.4835 GHZ and the second frequency band may include one or more channels in a Near-Field Communication (NFC) frequency band ranging from about 12.66 MHz to about 14.46 MHz.

The second antenna component 78 may include at least a Flexible Printed Circuit (FPC) or FPC board on which at least a portion of the second electrical circuit 78 resides. The first electrical circuit 80 may include a choke inductor that behaves like an open circuit when operating within the first frequency band and behaves like a short circuit when operating within the second frequency band. The second electrical circuit 82 may include blocking elements 96, 102, matching circuit elements 98, 104, and transceiver elements 100, 108 to enable operation within either the first frequency band or second frequency band. Also, the antenna system may further include one or more conductive strips and/or one or more ferrite strips attached to one or more of the first and second antenna components 76, 78.

In operation, the smart ring 50 uses the metal jacket or casing on the battery as part of the first antenna component 76 and can therefore serve as one of the arms of a dipole-like antenna, radiator, or transceiver. When the first electrical circuit 80 is shorted, the battery casing can serve as part of a current path for a loop antenna including both antenna components 76, 78. The battery can also serve as the ground plane of the antenna. In some embodiments, a thin metallic film (e.g., copper tape) can be installed along an outside surface of the battery.

The antenna may include, at least partially, one or more traces on the FPC board or PCB (i.e., flexible or rigid boards). Other parts of the antenna may include, at least partially, the metallization on the outside of the battery (e.g., battery case). A ground plane of the FPC may be the actual radiating element of the antenna, (e.g., no separate trace for the antenna element). Various techniques may be applied to protect the electronics from potentials that might be induced in the ground plane, disrupting their operation.

For the higher-frequency (Bluetooth, Wi-Fi) operation, the antenna has a dipole arrangement, but for the lower-frequency (NFC) operation, the antenna has a loop arrangement. The dipole can approximate a half wave dipole considering loading and tuning. The creation of either the dipole or loop arrangement can be determined by the state of the choke inductor 80. Also, the choke inductor 80 enables the antenna circuit to include higher-frequency or lower-frequency arrangements that can be tuned independently.

The metallic layer 74 of the smart ring 50 can be a parasitic element with a predetermined thickness. Also, the smart ring 50 may include a gap 92 between the metallic layer 74 and the first and second antenna components 76, 78. The gap 92 may have a predetermined width that can be designed to control the parasitic characteristics of the metallic layer 74.

The second electrical circuit 82 may include the capacitor 96 configured for isolation to protect the higher frequencies from the lower frequencies. Also, isolation by the inductor 102 can protect the lower frequency (NFC) circuits from the higher frequency signals.

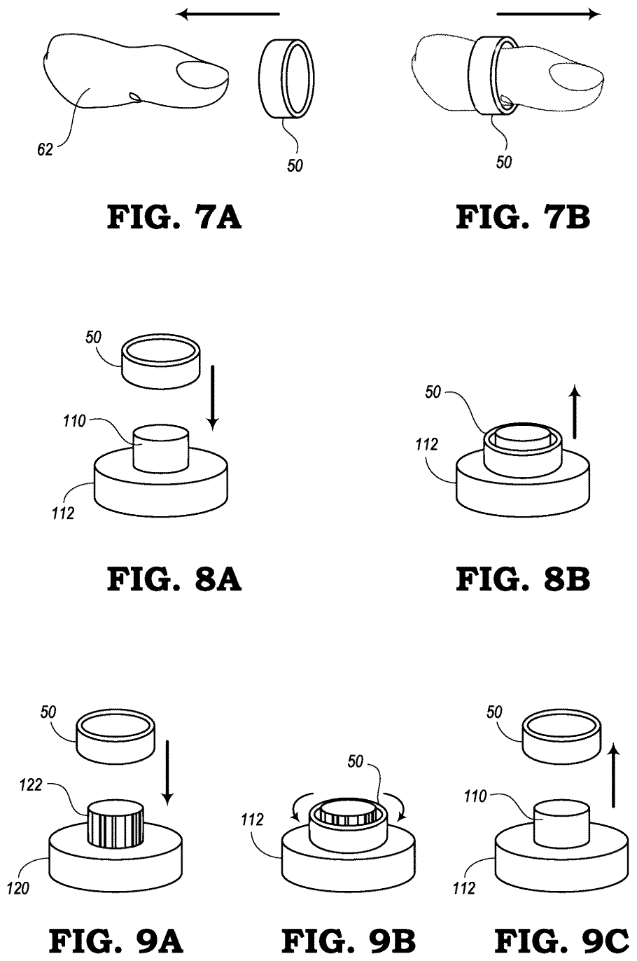

FIGS. 7A and 7B are diagrams illustrating detectable actions of placing the smart ring 50 on the user's finger and removing the smart ring from the user's finger 62. In this example, the smart ring 50 (e.g., wearable device 12) makes use of an existing sensor (e.g., internal sensor 18 configured as one or more capacitance sensors) to detect if the smart ring 50 is on the finger 62 (or on a charger or other component having a post). As such, processing device 20 (or decoding device) of the wearable device 12 or the smart ring 50 may use capacitance sensor to determine whether or not the smart ring 50 is on the finger 62 and output a binary 1 for “on” and a binary 0 for “off.” The processing device 20 may determine the binary numbers over a certain amount of time. When a specific on/off sequence is detected within a limited time, the processing device 20 may be configured to force the wireless communication device 22 to open the wireless communication link 25 with the secondary device 14. For example, a sequence of 01010101 (i.e., repeatedly moving the smart ring 50 on and off the finger 62 in a short amount of time) means that the user is entering a request to turn on the Bluetooth pairing mode. The last binary one may be an indication of an “on” request, while a last binary zero may be an indication of an “off” request for turning the Bluetooth pairing mode off.

FIGS. 8A and 8B are diagrams illustrating detectable actions of placing the smart ring 50 on a post 110 of a Near Field Communication (NFC) charger 112 and removing the smart ring 50 from the post 110 of the NFC charger 112. In this input method, the user may move the smart ring 50 on and off the post 110 repeatedly within a short amount of time. The smart ring 50 (or wearable device 12) may make use of an existing internal sensor 18 implemented as a wireless charger sensor (e.g., NFC sensor). The smart ring 50 may utilize the NFC sensor similar to the capacitance sensors mentioned above and/or detect the presence of NFC signals. The processing device 20 may output a binary one to indicate that the smart ring 50 is on the post 110 and a binary zero to indicated that the smart ring 50 is off the post 110. These codes may be same as described above or may be switched and may have a similar effect as a typical button press action for indicating whether to turn the Bluetooth pairing on or off. The particular sequence or pattern of codes could be user-defined or factory set codes.

FIGS. 9A-9C are diagrams illustrating the use of an image code for enabling the entry of a user request to change the state of the wireless communication link with the secondary device. In this embodiment, the internal sensor 18 of the smart ring 50 (or wearable device 12) may be implemented as an optoelectronic sensor (e.g., PPG sensor for detecting LED reflection). The output can be used to trigger the smart ring to turn on or turn off a Bluetooth pairing. The smart ring 50 may be used in association with a device 120 having a collar 122. Printed or applied to the collar 122 is an image code 124. In some embodiments, the device 120 may be an NFC charger and the collar 122 may be a charging post. The image code 124 may be a predetermined sequence of dark and light stripes (e.g., like a barcode or other type of scannable code). According to other embodiments in which the wearable device 12 includes another shape other than a ring, the image code 124 may be place on any suitable object to enable scanning.

As shown in FIG. 9A, the user first places the smart ring 50 on the collar 122, which may be configured to trigger the smart ring 50 to utilize a light source or LED to project light onto the image code 124. Also, an optoelectronic sensor for sensing light reflection can be activated. Then, as shown in FIG. 9B, the user rotates the smart ring 50 and the optoelectronic sensor is configured to detect the image code 124. For example, in some embodiments, the image code 124 may be readable (scannable) in both the clockwise direction and the counter-clockwise direction. For example, scanning in the counter-clockwise direction may be configured to produce an output that indicates a user request to open the wireless communication link 25, and scanning in the clockwise direction may be configured to produce an output that indicates a user request to close the wireless communication link 25, or vice versa. Based on the appropriate request, the processing device 20 is configured to cause the wireless communication device 22 to open or close the Bluetooth pairing link. Then, as indicated in FIG. 9C, the smart ring 50 can be removed.

### Processes

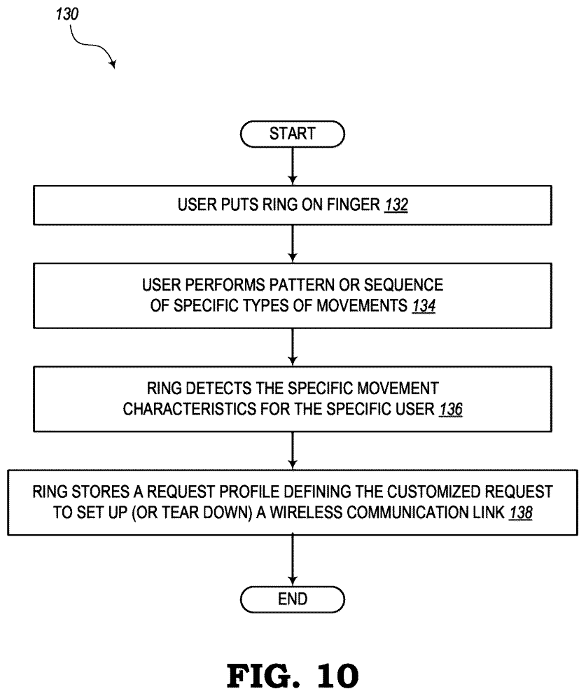

FIG. 10 is a flow diagram illustrating an embodiment of a process 130 for customizing a user request to change the state of a wireless communication link with a secondary device. In particular, the process 130 may apply to cases where one or more accelerometers are used to detect motion. As illustrated, the process 130 includes the step of allowing the user to place the ring on his or her finger, as indicated in block 132. This may include placing the smart ring 50 on the finger 62, or, alternatively, may include placing any type of wearable device (e.g., wearable device 12) on a corresponding part of the body of the user where movement patterns can be detected. The process 130 further includes allowing the user to perform a pattern or sequence of specific types of movement, as indicated in block 134. According to other embodiments, the process 130 may include movement detection with respect to an external object (e.g., finger 62, post 110, collar 122, etc.).

Next, the process 130 includes the step of detecting, by the smart ring, the specific movement characteristics for the specific user 136. Then, the process 130 includes storing, by the ring, a request profile defining the customized user request to set up (or tear down) a wireless communication link, as indicated in block 138. The process 130 may be performed once for the turn-on request profile customization and repeated for the turn-off request profile customization.

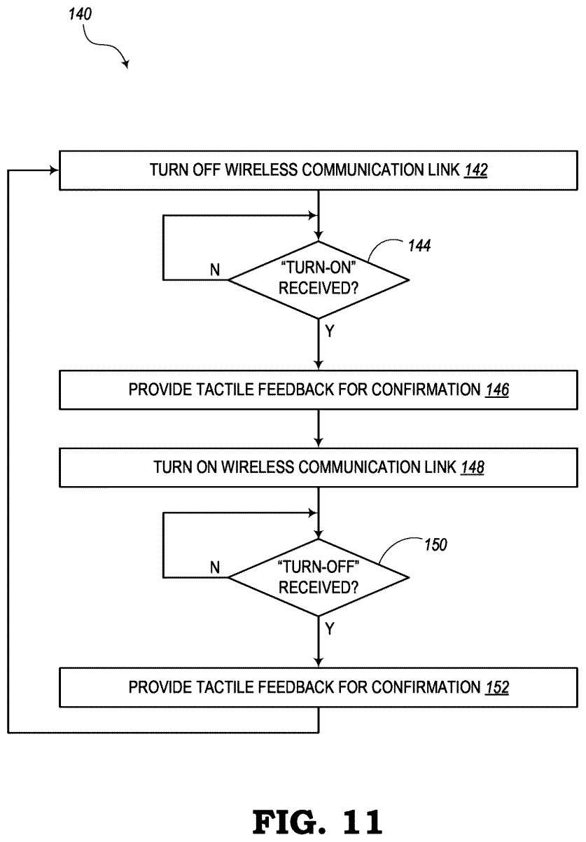

FIG. 11 is a flow diagram illustrating an embodiment of a process 140 for changing the state of the wireless communication link between a wearable device and a secondary device. Initially, the process 140 may include turning the wireless communication link off, as indicated in block 142. The process 140 then includes determining whether or not a request to turn-on the wireless communication link has been received, as indicated in condition diamond 144. If no such request is received, the state of the wireless communication link remains off. However, if a request to change the state is received, the process 140 proceeds to block 146, which includes the step of providing feedback (e.g., vibration) to the wearable device to indicate to the user that the request has been received. Also, the process 140 turns the wireless communication link on.

Then, the process 140 includes determining whether or not a request to turn-off the wireless communication has been received, as indicated in condition diamond 150. If no such request is received, the state of the wireless communication remains on. However, if a request to change the state is received, the process 140 proceeds to block 152, which includes the step of providing a feedback (e.g., vibration) to the wearable device to indicated to the user that the request has been received. Also, the process 140 loops back to block 142 and turns the wireless communication link off.

Therefore, according to the various embodiments of the present disclosure, a wearable device is provided, which includes a casing and an internal sensor arranged within the casing. The internal sensor is configured to electrically receive user input that is unrelated to manipulation of user-actuated mechanical components moveable with respect to the casing. In addition, the wearable device further includes a decoding device configured to decode the user input as a request to change the state of a wireless communication link with a secondary device.

In some embodiments, the wearable device may further comprise a wireless communication device configured to wirelessly communicate with the secondary device. The wireless communication device may be configured to wirelessly communicate with the secondary device in accordance with Bluetooth communication protocols. The step of changing the state of the wireless communication link may include setting up the wireless communication link between the wearable device and the secondary device or tearing down the wireless communication link between the wearable device and the secondary device. In response to the decoding device decoding the user input as an “on” request, the wireless communication device is configured to open up the wireless communication link with the secondary device. In response to the decoding device decoding the user input as an “off” request, the wireless communication device is configured to close the wireless communication link with the secondary device.

Furthermore, the internal sensor may include one or more capacitance sensors configured to detect capacitance for determining whether or not a user is wearing the wearable device. As such, the decoding device may be configured to decode multiple changes in capacitance within a predetermined time frame as a user request to change the state of the wireless communication link with the secondary device.

The internal sensor may also include one or more wireless charging sensors configured to sense the presence of Near Field Communication (NFC) signals for determining whether or not the wearable device is placed on an NFC charger. As such, the decoding device may be configured to decode multiple changes in the presence of NFC signals within a predetermined time frame as a user request to change the state of the wireless communication link with the secondary device.

In some embodiments, the internal sensor may include one or more optoelectronic sensors. As such, the decoding device may be configured to decode movement of the wearable device with respect to a predetermined code image as a user request to change the status of the wireless communication link with the secondary device. The predetermined code image, for example, may be applied to a post of a Near Field Communication (NFC) charger. As such, the decoding device may be configured to decode movement of the wearable device in a counter-clockwise direction around the post as a user request to open up the wireless communication link. Also, the decoding device may be configured to decode movement of the wearable device in a clockwise direction around the post as a user request to close the wireless communication link. In some cases, the one or more optoelectronic sensors may include a Photoplethysmography (PPG) sensor for measuring blood pressure of the user.

Also, the internal sensor may include one or more accelerometers configured to detect movement or vibrations of the wearable device or an external tapping action on a surface of the casing. As such, the decoding device may be configured to decode patterns of movements, vibrations, and/or tapping actions as user requests to change the state of the wireless communication link.

The wearable device may be a ring, a watch, glasses, smart glasses, earbuds, headphones, a bracelet, a necklace, a pendant, or other similar device. The internal sensor may be configured such that it includes no button components, switch components, or user-actuated mechanical components that can be physically accessible by a user. In some embodiments, the wearable device may further comprise a microphone and/or a camera for receiving supplemental user input and/or may include a speaker, a tone generator, a light source, and/or an LED for providing confirmation feedback to the user. The secondary device in these embodiments may be an access point device, a mesh node, a repeater, a local controller, or the like.

### CONCLUSION

It will be appreciated that some exemplary embodiments described herein may include one or more generic or specialized processors (“one or more processors”) such as microprocessors; Central Processing Units (CPUs); Digital Signal Processors (DSPs): customized processors such as Network Processors (NPs) or Network Processing Units (NPUs), Graphics Processing Units (GPUs), or the like; Field Programmable Gate Arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more Application-Specific Integrated Circuits (ASICs), in which each function or some combinations of certain of the functions are implemented as custom logic or circuitry. Of course, a combination of the aforementioned approaches may be used. For some of the exemplary embodiments described herein, a corresponding device in hardware and optionally with software, firmware, and a combination thereof can be referred to as “circuitry configured or adapted to,” “logic configured or adapted to,” etc. perform a set of operations, steps, methods, processes, algorithms, functions, techniques, etc. on digital and/or analog signals as described herein for the various exemplary embodiments.

Moreover, some exemplary embodiments may include a non-transitory computer-readable storage medium having computer readable code stored thereon for programming a computer, server, appliance, device, processor, circuit, etc. each of which may include a processor to perform functions as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), Flash memory, and the like. When stored in the non-transitory computer-readable medium, software can include instructions executable by a processor or device (e.g., any type of programmable circuitry or logic) that, in response to such execution, cause a processor or the device to perform a set of operations, steps, methods, processes, algorithms, functions, techniques, etc. as described herein for the various exemplary embodiments.

The foregoing sections include headers for various embodiments and those skilled in the art will appreciate these various embodiments may be used in combination with one another as well as individually. Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

What is claimed is:

1. A method comprising:
receiving, by a device, input, the input corresponding to an instruction for controlling a second device, the device and the second device electronically connected via a communication link; and
communicating, by the device, the instruction to the second device, the instruction causing the second device to change a state of operation, the state of operation of the second device corresponding to mechanisms for how the second device communicates with the device.

2. The method of claim 1, further comprising:
receiving instruction to sever the communication link between the device and the second device; and
halting a connection between the device and the second device in accordance with the instructions to sever the communication link.

3. The method of claim 2, further comprising the state of operation corresponding to the connection.

4. The method of claim 1, further comprising the state of operation corresponding to a type of the input.

5. The method of claim 1, further comprising the communication link being a type of Internet of Things (IoT) connection between the device and the second device.

6. The method of claim 1, further comprising detecting movement data via the first device, the input comprising the movement data.

7. The method of claim 1, further comprising the movement data comprising at least one of accelerometer data or gyroscope data.

8. The method of claim 1, further comprising the device being at least one of a mobile device, virtual reality (VR) device, smart device, heart monitoring device, wearable device or audio device.

9. The method of claim 1, further comprising the second device being at least one of a mobile device, access point device, gateway device, router, modem, speaker device or light device.

10. A device comprising:
a processor configured to:
receive input, the input corresponding to an instruction for controlling a second device, the device and the second device electronically connected via a communication link; and
communicate the instruction to the second device, the instruction causing the second device to change a state of operation, the state of operation of the second device corresponding to mechanisms for how the second device communicates with the device.

11. The device of claim 10, wherein the processor is further configured to:
receive instruction to sever the communication link between the device and the second device; and
halt a connection between the device and the second device in accordance with the instructions to sever the communication link.

12. The device of claim 11, wherein the processor is further configured such that the state of operation corresponds to the connection.

13. The device of claim 10, wherein the processor is further configured such that the state of operation corresponds to a type of the input.

14. The device of claim 10, wherein the processor is further configured such that the communication link is a type of Internet of Things (IoT) connection between the device and the second device.

15. The device of claim 10, wherein the processor is further configured to detect movement data via the first device, the input comprising the movement data.

16. The device of claim 10, wherein the processor is further configured such that the movement data comprises at least one of accelerometer data or gyroscope data.

17. The device of claim 10, wherein the processor is further configured such that the device is at least one of a mobile device, virtual reality (VR) device, smart device, heart monitoring device, wearable device or audio device.

18. The device of claim 10, wherein the processor is further configured such that the second device is at least one of a mobile device, access point device, gateway device, router, modem, speaker device or light device.

19. A method comprising:
receiving, by a second device, instruction from a first device, the instruction comprising information for controlling the second device, the first device and the second device electronically connected via a communication link; and
causing, via the second device, a change to a state of operation of the second device, the state of operation of the second device corresponding to mechanisms for how the second device communicates with the first device.

20. The method of claim 19, further comprising the instruction comprising an instruction to sever the communication link between the first device and the second device, wherein the method further comprises halting a connection between the first device and the second device.
